SEQUENCE LISTING (7 sequences)

SEQ ID NO: 1 - DNA, 23 bp, synthetic construct
  misc_feature 1..23: note = synthetic oligonucleotide
  source 1..23: mol_type = other DNA
gcagcatgtcaagatcacagatt

SEQ ID NO: 2 - DNA, 25 bp, synthetic construct
  misc_feature 1..25: note = synthetic oligonucleotide
  source 1..25: mol_type = other DNA
cctccttctgcatggtattctttct

SEQ ID NO: 3 - DNA, 26 bp, synthetic construct
  misc_feature 1..26: note = synthetic oligonucleotide
  source 1..26: mol_type = other DNA
tttgggcaggccaaactgctgggtgc

SEQ ID NO: 4 - DNA, 26 bp, synthetic construct
  misc_feature 1..26: note = synthetic oligonucleotide
  source 1..26: mol_type = other DNA
tttggcctgcccaaaatctgtgatct

SEQ ID NO: 5 - DNA, 26 bp, synthetic construct
  misc_feature 1..26: note = synthetic oligonucleotide
  source 1..26: mol_type = other DNA
tttgggccggccaaactgctgggtgc

SEQ ID NO: 6 - DNA, 26 bp, synthetic construct
  misc_feature 1..26: note = synthetic oligonucleotide
  source 1..26: mol_type = other DNA
tttggccggcccaaaatctgtgatct

SEQ ID NO: 7 - DNA, 52 bp, synthetic construct
  misc_feature 1..52: note = synthetic oligonucleotide
  source 1..52: mol_type = other DNA
caagctatttaggtgacactatagcagcatgtcaagatcacagattttgggc

(19) United States
(12) Patent Application Publication
(10) Pub. No.: US 2025/0341468 A1
(43) Pub. Date: Nov. 6, 2025

(54) GENE ANALYSIS METHOD, GENE ANALYSIS APPARATUS, AND GENE ANALYSIS KIT

(71) Applicant: HITACHI, LTD., Tokyo (JP)

(72) Inventors: Takahiro Ando, Tokyo (JP); Takahide Yokoi, Tokyo (JP); Chihiro Manri, Tokyo (JP); Takashi Anazawa, Tokyo (JP); Shoko Kawakami, Tokyo (JP); Takeshi Ishida, Tokyo (JP)

(73) Assignee: HITACHI, LTD., Tokyo (JP)

(21) Appl. No.: 18/857,536

(22) PCT Filed: Feb. 20, 2023
(86) PCT No.: PCT/JP2023/006013

§ 371 Date: Oct. 17, 2024

(30) Foreign Application Priority Data
Jun. 17, 2022 (JP) 2022-097908

(51) Int. Cl. G01N 21/64; G01N 27/447

(52) U.S. Cl. CPC G01N 21/6486; G01N 27/44726; G01N 2201/125

(57) ABSTRACT

The present invention relates to a gene analysis method which includes: conducting a single base extension reaction with use of a single base extension primer for detecting a target base sequence, and a fluorescent dye-labeled substrate for single base extension; subjecting a reaction product of the single base extension reaction to electrophoresis; and measuring mobility in the electrophoresis and fluorescence intensity of the fluorescent dye, and quantifying a content ratio of a plurality of target base sequences from a magnitude of the fluorescence intensity, wherein in the single base extension reaction, a fluorescent dye-free substrate for single base extension is mixed.

### SEQUENCE LISTING

The instant application contains a Sequence Listing which has been submitted electronically in XML format and is hereby incorporated by reference in its entirety. Said XML copy, created on Oct. 8, 2024, is named 0723881679SL.xml and is 9,899 bytes in size.

### TECHNICAL FIELD

The present invention relates to a gene analysis method for conducting quantitative analysis of genetic mutation with use of a single base extension reaction, a gene analysis apparatus based on the method, and a gene analysis kit.

### BACKGROUND ART

The dideoxy method developed by Sanger et al. has been known as a method of DNA sequencing. DNA to be analyzed is introduced into a vector, amplified, and denatured to produce single-stranded template DNA. A primer is bound to the template DNA, thereby starting complementary strand synthesis from the primer. In this process, one specific kind of dideoxynucleotide triphosphate which acts as a terminator is preliminarily added, besides four types of deoxynucleotide triphosphates. The complementary strand synthesis will stop upon incorporation of the dideoxynucleotide triphosphate (ddNTP), whereby DNA fragments with various lengths terminated by the specific bases are obtainable. With use of each of the dideoxynucleotide triphosphates corresponded to four types of bases of adenine (A), cytosine (C), guanine (G), and thymine (T), that is, ddATP, ddCTP, ddGTP, and ddTTP, respectively, the aforementioned complementary strand synthesis reaction is allowed to proceed, to obtain DNA fragments having various lengths whose terminal bases are A, C, G and T, respectively, and the obtained DNA fragments are then subjected to molecular weight separation. The base sequence can be analyzed by reading the base types in the order of the molecular weight. The molecular weight separation may rely upon polyacrylamide gel electrophoresis, or capillary electrophoresis.

A DNA sequencer making use of capillary electrophoresis is an apparatus for analyzing a base sequence, by subjecting a DNA sample, labeled with four colors of florescent dye, to electrophoresis in a capillary. Owing to adaptability to continuous automatic analysis, and ability of high-speed and parallel multi-sample analysis, the apparatus has greatly contributed to large-scale gene sequencing such as in the Human Genome Project, and has been widely used up to now as a most reliable methodology. Determination of the base sequence of DNA sample involves, in principle, electrophoretic separation of DNA strands by length, and detection of fluorescently labeled ddNTP at positions of separation. The base estimation from the obtained fluorescence signal intensity is determined by majority decision at each peak coordinate position, with reference to the signal intensity, or the area of the signal waveform. This enables accurate decision of base sequence, with no need for considering difference in fluorescence intensity per se among four colors of fluorescent labels corresponded to the terminal bases A, C, G, and T. On the other hand, PTL 1 proposes a method of determining A, C, G and T, by utilizing difference in fluorescence intensity characteristics of such four colors of fluorescent label.

Recent advancement in cancer research has raised importance of detecting tumor-derived genetic mutations, with use of technique of genetic analysis. In particular, a test designed to detect tumor-derived genetic mutation in blood for medical diagnosis, called liquid biopsy, has been expected for applications for example in early diagnosis of cancer, assistance for best choice of post-surgery therapy, and monitoring of residual tumor. In the current scene of the search for tumor-associated genetic mutation that serves as a biomarker, a next generation sequencer (NGS) has enabled large-scale and high-speed analysis, making it easier to extract items of the genetic mutation necessary for liquid biopsy. A current trend in cancer diagnosis, for example, is directed to enhancement of versatility of a test technique, by specifying tumor-derived genetic mutation with reference to results of comprehensive analysis with NGS, followed by measurement by a genetic mutation detection technique which is advantageous over NGS in terms of cost and detection sensitivity.

One known low-cost and high-sensitivity technique for detecting genetic mutation is exemplified by fragment analysis that employs capillary electrophoresis. As illustrated in FIG. 1, the method is designed to use selective primers with different molecular weights adapted to each of target gene sequences so as to differentiate electrophoretic mobility, and to add ddNTPs labeled with four types of fluorescent dye to the 3′ end position of the selective primer that corresponds to the genetic mutation, as a result of single base extension reaction based on polymerase synthesis reaction. The double-stranded DNA is then subjected to formamide treatment and thermal denaturation to obtain single strand. Genetic mutation is determined by detecting fluorescence of the fluorescent dye at the 3′ end. With use of this method, NPL 1, for example, has detected 120 known genetic mutations in 13 cancer genes from a tumor-derived target gene sequence, after selective concentration by a multiplex polymerase chain reaction (PCR). Since, however, the migration length, at which the selective primers are electrophoretically separable, is limited up to 120 bases, so that only several types of genetic mutation have been separable at a time per run. Moreover, fluorescence intensity varies among the fluorescent dyes for specifying the genetic mutation. The present inventors have recently succeeded in increasing the number of genetic mutations detectable at a time, by binding double-stranded DNAs having inter-strand crosslinks to the selective primers to stably extend the electrophoretic migration distance up to 120 bp or beyond. An electrophoretic region having not been effectively utilized now became available.

### CITATION LIST

### Patent Literature

PTL 1: JP H05-118991 A (1993)

### Non-Patent Literature

NPL 1: Dias-Santagata, D. et al., EMBO Molecular Medicine, Vol. 2, p. 146-158 (2010)

### SUMMARY OF INVENTION

### Technical Problem

A possible example of low-cost and high-sensitivity cancer diagnosis with use of liquid biopsy would be fragment analysis based on capillary electrophoresis, capable of detecting 100 or more types of known genetic mutation. The process of examining how many mutants are expressed as compared with a wild-type gene that expresses a normal state has, however, failed in quantitatively determining the ratio of wild-type to mutant affected by detection sensitivity, since the aforementioned conventional technique of detecting genetic mutation based on single base extension has suffered from difference in fluorescence excitation efficiency among the fluorescent dyes, and difference in signal intensity as a consequence.

### Solution to Problem

After intensive studies aimed at solving the aforementioned problems, the present inventors found that the content ratio of target base sequences (wild type and mutant, for example) can be quantitatively determined from the magnitude of fluorescence intensity, by taking a special approach of mixing a fluorescent dye-free substrate for single base extension, in the single base extension reaction that uses a single base extension primer for detecting a target gene sequence, and a fluorescent dye-labeled substrate for single base extension. The mixing ratio of the fluorescent dye-free substrate may be set according, for example, to ratio of excitation efficiency of the fluorescent dye to be detected, or binding-and-uptake efficiency.

In one aspect, the present invention relates to a gene analysis method which includes:
conducting a single base extension reaction with use of a single base extension primer for detecting a target base sequence, and a fluorescent dye-labeled substrate for single base extension;
subjecting a reaction product of the single base extension reaction to electrophoresis; and
measuring mobility in the electrophoresis and fluorescence intensity of the fluorescent dye, and quantifying a content ratio of a plurality of target base sequences from a magnitude of the fluorescence intensity,
wherein in the single base extension reaction, a fluorescent dye-free substrate for single base extension is mixed, and
wherein a mixing ratio of the fluorescent dye-free substrate and the fluorescent dye-labeled substrate is set according to:
(a) ratio of excitation efficiency of the fluorescent dyes to be detected, in a case where at least two types of fluorescent dye are used; and/or
(b) binding-and-uptake efficiency of the substrate to the primers to be used, in a case where at least two types of primer are used.

In another aspect, the present invention relates to a gene analysis apparatus which includes:
a measurement unit configured to measure single base extension reaction, electrophoresis, and fluorescence intensity;
a data analysis unit comprising a measurement data storage unit configured to store measurement data obtained by the measurement unit, and a data processing unit; and
a control unit;
wherein the control unit is configured to analyze the measurement data stored in the measurement data storage unit, and to determine a mixing ratio of a fluorescent dye-labeled substrate and a fluorescent dye-free substrate to be used in the single base extension reaction.

In still another aspect, the present invention relates to a gene analysis kit which includes:
a single base extension primer for detecting a target base sequence;
a fluorescent dye-labeled substrate for single base extension; and
a fluorescent dye-free substrate for single base extension,
wherein a content ratio of the fluorescent dye-free substrate and the fluorescent dye-labeled substrate is set according to:
(a) ratio of excitation efficiency of the fluorescent dyes to be detected, in a case where at least two types of fluorescent dye are used; and/or
(b) binding-and-uptake efficiency of the substrate to the primers to be used, in a case where at least two types of primer are used.

This specification contains the disclosure of Japanese Patent Application No. 2022-097908 filed on Jun. 17, 2022, based on which the present application claims priority.

### Advantageous Effects of Invention

The present invention can quantitatively determine the content ratio of target gene sequences from the magnitude of fluorescence intensity, and can quantitatively determine the abundance ratio of genetic mutation to wild-type, or frequency of the genetic mutation, which is particularly required for cancer diagnosis. Problems, structures, and effects other than those described above will be clarified by the following description of embodiments.

### BRIEF DESCRIPTION OF DRAWINGS

FIG. 1 is an explanatory drawing illustrating a fragment analysis technique based on capillary electrophoresis.

FIG. 2 is an explanatory drawing illustrating that use of ddNTPs labeled with fluorescent dyes differed for each tumor-derived target gene sequence will result in different relative fluorescent intensities.

FIG. 3 is an explanatory drawing illustrating that uptake efficiency of fluorescent dye-labeled ddNTP will differ among the tumor-derived target gene sequences.

FIG. 4 is an explanatory drawing illustrating an exemplary solving means of the present invention, taking a special approach of using a fluorescent dye-free ddNTP, in the process of using the ddNTPs labeled with fluorescent dyes differed for each tumor-derived target gene sequence, so as to make the relative fluorescence intensity quantitative.

FIG. 5 shows results of template concentration and peak values of fluorescence intensity, before and after the use of the fluorescent dye-free ddNTPs.

FIG. 6 is a flowchart illustrating an exemplary procedure of processes with use of a gene analysis apparatus and a gene analysis kit for implementing the present invention.

FIG. 7 is a block structural diagram illustrating an exemplary function installed on the gene analysis apparatus of the present invention.

### DESCRIPTION OF EMBODIMENTS

Hereinafter, an exemplary embodiment of the present invention will be described while referring to the attached drawings.

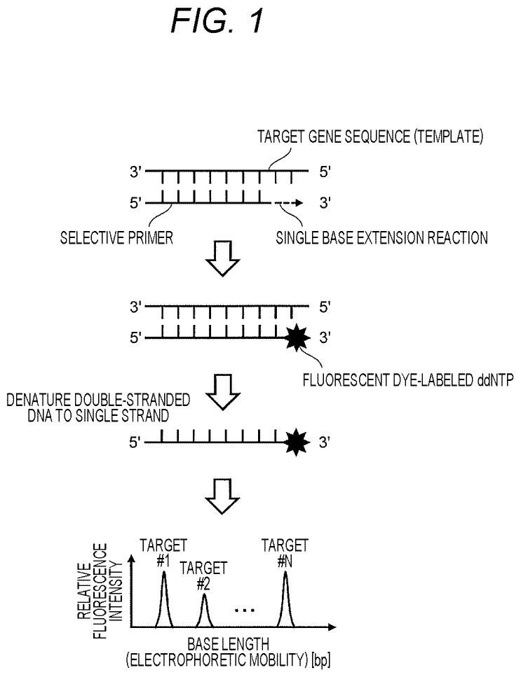

As described previously while referring to FIG. 1, the present invention employs fragment analysis based on capillary electrophoresis. A widely accepted fragment analysis is designed to use selective primers with different molecular weights adapted to each of target gene sequences so as to differentiate electrophoretic mobility, and to add ddNTPs labeled with four types of fluorescent dye to the 3′ end position of the selective primer that corresponds to the genetic mutation, as a result of single base extension reaction based on polymerase synthesis reaction on a target gene sequence used as a template. The double-stranded DNA is converted to single strands by the formamide treatment and thermal denaturation, and fluorescence of the fluorescent dye at the 3′ end is detected, thereby determining the genetic mutation. Use of selective primers having different molecular weights enables detection of 100 or more types of known genetic mutation. In this process with a properly designed primer, not only a gene sequence having a specific length, but also a single base polymorphism having only a single base mutation, is detectable. Alternatively, since also insertion and deletion, which are sorts of genetic mutation, are detectable on the same principle, the applicable range of the present invention can entirely cover the fragment analysis with use of the fluorescent dye-labeled ddNTP.

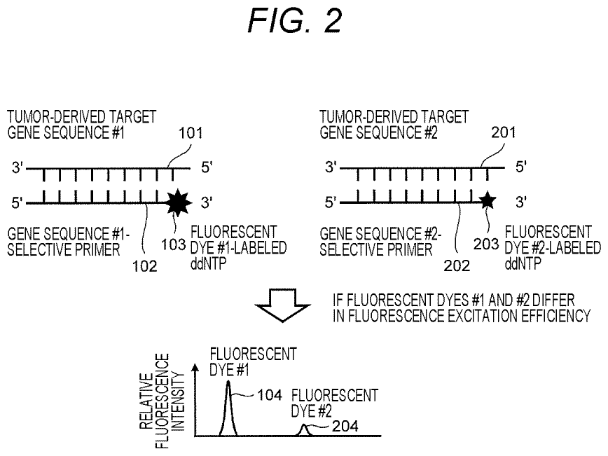

FIG. 2 is an explanatory drawing illustrating that use of ddNTPs labeled with fluorescent dyes differed for each tumor-derived target gene sequence will result in different relative fluorescent intensities. On the tumor-derived target gene sequence #1 indicated by 101, a fluorescent dye #1-labeled ddNTP 103 is added to the 3′ terminal position of a gene sequence #1-selective primer 102, as a result of single base extension reaction based on polymerase synthesis reaction, according to the principle illustrated in FIG. 1. Similarly, on a tumor-derived target gene sequence #2 indicated by 201, a fluorescent dye #2-labeled ddNTP 203 is added to the 3′ terminal position of a gene sequence #2-selective primer 202, as a result of single base extension reaction. If the fluorescent dye #2 herein demonstrates smaller fluorescence excitation efficiency than the fluorescent dye #1, relative fluorescence intensity 204 ascribed to the fluorescent dye #2 will be smaller than relative fluorescence intensity 104 ascribed to the fluorescent dye #1. Although being dependent typically on reagent environment (mixture, temperature, pH, etc., for example) during measurement, or on electrophoresis conditions (injection voltage, injection rate, electrophoresis voltage, temperature, etc., for example) of the measurement apparatus, or on excitation wavelength, the fluorescence excitation efficiency may be preliminarily measured taking measurement conditions into account, thus making it possible to preliminarily prepare data on to what extent the relative fluorescence intensity will differ.

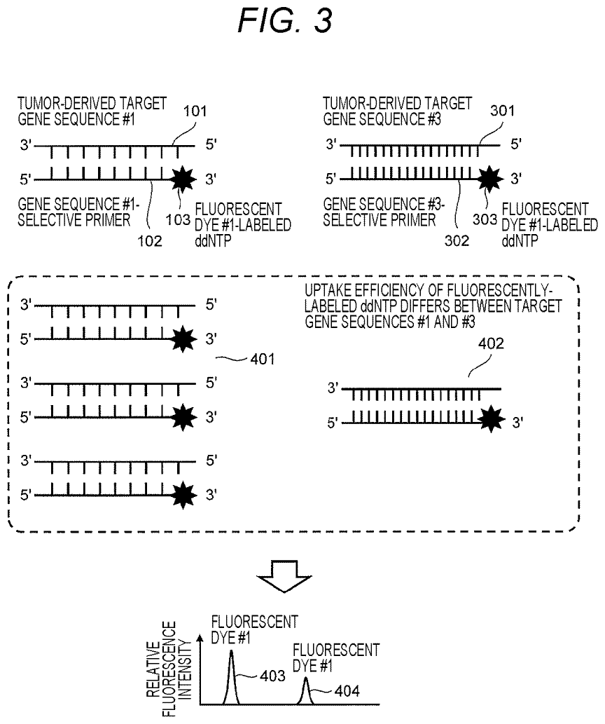

FIG. 3 is an explanatory drawing illustrating that uptake efficiency of fluorescent dye-labeled ddNTP will differ among the tumor-derived target gene sequences (primers). On the tumor-derived target gene sequence #1 indicated by 101, a fluorescent dye #1-labeled ddNTP 103 is added to the 3′ terminal position of a gene sequence #1-selective primer 102, as a result of single base extension reaction. Similarly, on a tumor-derived target gene sequence #3 indicated by 301, a fluorescent dye #1-labeled ddNTP 303 is added to the 3′ terminal position of a gene sequence #3-selective primer 302, as a result of single base extension reaction. Now, if there are a state 401 representing high uptake efficiency of the fluorescent-labeled ddNTP on the gene sequence #1, and a state 402 representing low uptake efficiency of the fluorescently-labeled ddNTP on the gene sequence #3, observed is relative fluorescence intensity 404 ascribed to the fluorescent dye #1 under low uptake efficiency of the fluorescently-labeled ddNTP on the gene sequence #3, which is smaller than relative fluorescence intensity 403 ascribed to the fluorescent dye #1 under high uptake efficiency of the fluorescently-labeled ddNTP on the gene sequence #1. Although being dependent on combination with the selective primer, or on reagent environment (mixture, temperature, pH, etc., for example) during the measurement, irrespective of whether or not having fluorescence labeling, the uptake efficiency of ddNTP may be preliminarily measured taking measurement conditions into account, thus making it possible to preliminarily prepare data on to what extent the uptake efficiency of ddNTP will differ.

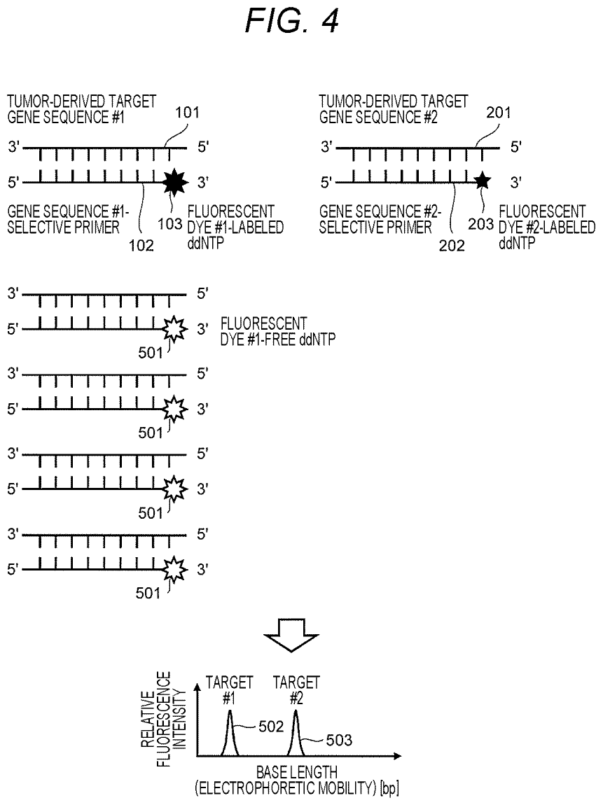

FIG. 4 is an explanatory drawing illustrating an exemplary solving means of the present invention, taking a special approach of using a fluorescent dye-free ddNTP, in the process of using the ddNTPs labeled with fluorescent dyes differed for each tumor-derived target gene sequence, so as to make the relative fluorescence intensity quantitative. On the tumor-derived target gene sequence #1 indicated by 101, a fluorescent dye #1-labeled ddNTP 103 is added to the 3′ terminal position of a gene sequence #1-selective primer 102, as a result of single base extension reaction based on polymerase synthesis reaction, according to the principle illustrated in FIG. 1. Similarly, on a tumor-derived target gene sequence #2 indicated by 201, a fluorescent dye #2-labeled ddNTP 203 is added to the 3′ terminal position of a gene sequence #2-selective primer 202, as a result of single base extension reaction. If, for example, fluorescent dye #2 herein demonstrates smaller fluorescence excitation efficiency than the fluorescent dye #1, addition of the fluorescent dye #1-free ddNTP 501 to the single base extension reagent will lower the relative fluorescence intensity than in a case without the addition. Accordingly, in such state with use of the fluorescent dye #1-free ddNTP, it becomes possible to correct relative fluorescence intensity 502 ascribed to the fluorescent dye #1 representing the abundance of the gene sequence #1, to be equalized to relative fluorescence intensity 503 ascribed to the fluorescent dye #2 representing the abundance of the gene sequence #2. That is, quantitativeness in the ratio of abundance between the gene sequence #1 and the gene sequence #2 may be ensured. This makes it possible to clarify to what extent the mutant is expressed, relative to the wild-type gene in the normal state. This is adjustable not only in a case where the fluorescence excitation efficiency differs, but also in a case where the ddNTP uptake efficiency differs, making it possible to quantitatively determine the ratio of the wild type and the mutant.

Accordingly in one aspect, the present invention is to provide a gene analysis method, the method includes:
conducting a single base extension reaction with use of a single base extension primer for detecting a target base sequence, and a fluorescent dye-labeled substrate for single base extension;
subjecting a reaction product of the single base extension reaction to electrophoresis; and
measuring mobility in the electrophoresis and fluorescence intensity of the fluorescent dye, and quantifying a content ratio of a plurality of target base sequences from a magnitude of the fluorescence intensity,
wherein in the single base extension reaction, a fluorescent dye-free substrate for single base extension is added, and
wherein a mixing ratio of the fluorescent dye-free substrate and the fluorescent dye-labeled substrate is set according to:
(a) ratio of excitation efficiency of the fluorescent dyes to be detected, in a case where at least two types of fluorescent dye are used; and/or
(b) binding-and-uptake efficiency of the substrate to the primers to be used, in a case where at least two types of primer are used.

The present invention is based on a gene analysis method making use of combination of single base extension reaction and electrophoresis. This sort of gene analysis method has been known in the art, as described for example in NPL 1.

The single base extension reaction may be conducted with use of a single base extension primer for detecting target base sequence, in the presence of a fluorescent dye-labeled substrate (dideoxynucleotide triphosphate). Meanwhile, the present invention allows the single base extension reaction to proceed also involving the fluorescent dye-free substrate in the reaction, in order to correct the ratio of fluorescence intensity due to difference in excitation efficiency of the fluorescent dyes, and/or uptake efficiency of the substrate into the primer.

The test sample subjected to the present method may not be particularly limited as long as whose target base sequence is to be detected, and includes deoxyribonucleic acid (DNA) such as genomic DNA and cDNA, and ribonucleic acid (RNA) such as messenger RNA (mRNA) and fragments thereof. According to the present invention, cell-free DNA (cfDNA, which is free DNA in blood), or circulating tumor DNA (ctDNA) may preferably be used as the test sample. Nucleic acid may be prepared from the sample, by any of methods known in the art. A lot of manufactures have marketed kits for preparing nucleic acid, with which a desired nucleic acid may be purified easily.

A single base extension primer may be prepared. The single base extension primer may be either DNA or RNA, which is determined according to the types of the test sample and the target base sequence, and to the type of polymerase used in the single base extension reaction. The primer may preferably be DNA, with which the single base extension is allowed to proceed with use of DNA or mRNA as the test sample that serves as the template.

The primer may be designed so as to have a sequence that can specifically bind to the target base sequence, that is, to have a sequence complementary to the target base sequence. Technique for designing the primer has been known in the art. The primer which can be used according to the present invention may be designed so as to satisfy conditions under which specific annealing can proceed, for example, so as to have a length and base composition (melting temperature) allowed for specific annealing. For example, the primer capable of functioning preferably has a length of 10 bases or longer, which is more preferably 15 to 50 bases, even more preferably 15 to 30 bases, and typically approximately 20 bases. When designed, the primer may preferably be checked in terms of GC content and the melting temperature (Tm). Tm may be checked with use of any of known software for primer design. The thus designed primer, although chemically synthesizable by any of known methods for synthesizing oligonucleotide, may usually be synthesized with use of a commercially available chemical synthesis apparatus.

The primer may have a double-stranded DNA tag having an inter-strand crosslink. The present inventors have previously developed an analysis technique which was advanced from the fragment analysis method based on capillary electrophoresis, so as to expand the number of genetic mutations detectable at a time up to several tens to several hundreds of types. More specifically, the present inventors have successfully increased the number of genetic mutations detectable at a time, by binding a double-stranded DNA tag having at least one inter-strand crosslink to the primer, thereby stably extending the electrophoretic migration distance up to 120 bp or beyond, as a result of change in the length of the double-stranded DNA tag. The double-stranded DNA tag has a length that is distinguishable by mobility, and has at least one inter-strand crosslink. In the present invention, “inter-strand crosslink” means that one strand and the other strand in the double-stranded DNA are crosslinked at least at one point. Method for making such intra-molecular crosslink between two strands may not be particularly limited as long as it has been known in the art. The inter-strand crosslink is preferably formed by photo-crosslinking. The double-stranded DNA tag having an inter-strand crosslink defines a migration distance (mobility) in electrophoresis. That is, binding with the double-stranded DNA tags having different lengths to the primers can change the migration distance in electrophoresis. Since the capillary electrophoresis can detect nucleic acid having a length of up to approximately 600 bases, so that the double-stranded DNA tag may have a length of one to approximately 590 bases, excluding the length of the primer (10 to 30 bases) to be bound to the target base sequence. The double-stranded DNA tag may not be particularly limited in terms of base sequence, as long as it is a nucleic acid having an inter-strand crosslink. The double-stranded DNA tag, although chemically synthesizable by any of known methods for synthesizing oligonucleotide, may usually be synthesized with use of a commercially available chemical synthesis apparatus.

In the method of the present invention, the single base extension reaction is allowed to proceed with use of the aforementioned primer, in the presence of the aforementioned fluorescent dye-labeled substrate and the fluorescent dye-free substrate. The single base extension reaction has been known in the art, which typically uses a polymerase. The polymerase used therefor may be selected depending on types of the template (test sample) and types of the primer to be used. For an exemplary single base extension reaction with use of DNA or RNA as a template, employed is a DNA-dependent DNA polymerase or an RNA-dependent DNA polymerase, respectively.

The single base extension reaction has been widely known in the art. For example, NPL 1 typically explains a method that uses a cycle reaction, enabling efficient single base extension.

In the presence of a target base sequence, the primer hybridizes to the target base sequence, and a nucleotide is incorporated at the 3′ end of the primer, as a result of polymerase synthesis reaction. With use herein of, for example, a dideoxynucleotide (ddNTP) as a nucleotide (substrate) to be incorporated, the synthesis reaction may stop after extension by only one base.

According to the present invention, a fluorescent dye-labeled substrate and a fluorescent dye-free substrate may be used as such substrate. The fluorescent dye may be useful for conveniently detecting whether or not the substrate has been incorporated, or for determining the type of base incorporated, for which any of fluorescent dyes known in the art is applicable. The fluorescent dye may include, but not limited to, fluorescein, fluorescein isothiocyanate (FITC), sulforhodamine (TR), tetramethylrhodamine (TRITC), carboxy-X-rhodamine (ROX), carboxytetramethylrhodamine (TAMRA), NED, 5-carboxyfluorescein (5-FAM), 6-carboxyfluorescein (6-FAM), 5′-hexachlorofluorescein CE-phosphoramidite (HEX), 6-carboxy-4′,5′-dichloro-2′,7′-dimethoxyfluorescein (JOE), 5′-tetrachlorofluorescein CE-phosphoramidite (TET), rhodamine 110 (R110), rhodamine 6G (R6G), VIC (registered trademark), ATTO dyes, and Alexa Fluor (registered trademark), Texas red, and Cy dies. The fluorescent dye that does not cause shift of phoretic size may include dR110 (carboxy-dichlororhodamine 110), dR6G (dihydro rhodamine 6G), dTAMRA (tetramethyl rhodamine), and dROX (carboxy-X-rhodamine). When trying to determine the types of base, five types of fluorescent dyes that are excited and detected at different wavelengths may be used in combination, in order to identify four types of base and a reference base (for detecting and correcting the base length from a reference ladder DNA), totaling five types. The types of the fluorescent dye, and methods of incorporation used herein may be any of various known means, without special limitation.

The mixing ratio of the fluorescent dye-free substrate to the fluorescent dye-labeled substrate may be set according to:
(a) the ratio of excitation efficiency of the fluorescent dyes to be detected, in a case where at least two types of fluorescent dye are used; and/or
(b) the binding-and-uptake efficiency of the substrate to the primers to be used, in a case where at least two types of primer are used.

In an exemplary case, as illustrated in FIG. 2, where the fluorescent dye includes at least two types of fluorescent dye (individually differed in the fluorescence excitation efficiency), the mixing ratio of the fluorescent dye-free substrate and the fluorescent dye-labeled substrate may be set according to the ratio of excitation efficiency of the fluorescent dyes to be detected. More specifically, the single base extension reaction is allowed to proceed, by adding a fluorescent dye-free substrate in which the substrate is the same one labeled with the fluorescent dye having higher fluorescence excitation efficiency. The mixing ratio of the fluorescent dye-free substrate and the fluorescent dye-labeled substrate may be set according to previous measurement data, or according to an optimum mixing ratio determined by a preliminary experiment before the actual gene analysis. In another exemplary case where four types of fluorescent dye corresponded to four types of base are used, the genes that incorporated the individual bases may be quantitatively analyzed, by determining a mixing ratio of the fluorescent dye-free substrate and the fluorescent dye-labeled substrate for each of the four types of fluorescent dye, and by measuring the fluorescence signal intensities of the four types of fluorescent dye.

In an exemplary case, as illustrated in FIG. 3, where the primer includes at least two types of primer (that is, at least two types of primer corresponded to at least two types of target base sequence, with different binding-and-uptake efficiencies of the substrate to the primers), the mixing ratio of the fluorescent dye-free substrate and the fluorescent dye-labeled substrate may be set according to the binding-and-uptake efficiency of the substrate to the primers to be used. More specifically, the single base extension reaction is allowed to proceed, by adding a fluorescent dye-free substrate in which the substrate is the same one labeled with the fluorescent dye having higher binding-and-uptake efficiency. The mixing ratio of the fluorescent dye-free substrate and the fluorescent dye-labeled substrate may be set according to previous measurement data, or according to an optimum mixing ratio determined by a preliminary experiment before the actual gene analysis.

In another exemplary case where the primer includes at least two types of primer, and the fluorescent dye includes at least two types of fluorescent dye, the mixing ratio of the fluorescent dye-free substrate and the fluorescent dye-labeled substrate may be set according to the ratio of excitation efficiency of the fluorescent dyes to be detected, and to the binding-and-uptake efficiency of the substrates to the primers to be used.

After the single base extension reaction, the obtained reaction product is analyzed by subjecting it to electrophoresis, which is more preferably capillary electrophoresis (CE). The electrophoresis, which is typically CE, is a technique of separating injected components according to difference in mobility ascribed to electric charge, size, or shape, for example. Types of the target base sequence (based on the types of primer) may be identified with reference to the mobility. Also, presence or absence of the target base sequence, or the types of the specific base in the target base sequence (based on the types of the substrate incorporated by the single base extension reaction) may be determined with reference to signals of the fluorescent dyes.

As described above, the present invention can quantitatively determine the content ratio of the plurality of target base sequences from the magnitude of the fluorescence intensity, by conducting the single base extension reaction while mixing the fluorescent dye-labeled substrate and the fluorescent dye-free substrate. The present invention can therefore quantify the ratio of abundance of mutant sequence to wild-type sequence necessary for cancer diagnosis, or frequency of genetic mutation. In one embodiment, the target base sequence may be quantified, in a case where a plurality of target base sequences to be analyzed contain wild-type sequence and mutant sequence, and the content ratio of the mutant sequence to the wild-type sequence may be in the range of 0.01% to 1%, which is typically 0.01% to 0.1%. Quantitative gene analysis for the target base sequence may thus be enabled.

The aforementioned gene analysis method of the present invention may be implemented easily and quickly, with use of a gene analysis apparatus equipped with necessary structures, or with use of a gene analysis kit that contains necessary constituents.

Another aspect of the present invention therefore provides a gene analysis apparatus which includes:
a measurement unit configured to measure single base extension reaction, electrophoresis, and fluorescence intensity;
a data analysis unit including a measurement data storage unit configured to store measurement data obtained by the measurement unit, and a data processing unit; and
a control unit;
wherein the control unit is configured to analyze the measurement data stored in the measurement data storage unit, and to determine a mixing ratio of a fluorescent dye-labeled substrate and a fluorescent dye-free substrate to be used in the single base extension reaction.

The control unit may further include a reference database configured to store previous measurement data. The control unit in this case may be configured to compare the measurement data stored in the measurement data storage unit, with the previous measurement data stored in a reference database, and to determine the mixing ratio of the fluorescent dye-labeled substrate and the fluorescent dye-free substrate to be used in the single base extension reaction.

The gene analysis apparatus of the present invention may further include an output display unit.

In still another aspect, the present invention provides a gene analysis kit which includes:
a single base extension primer for detecting a target base sequence;
a fluorescent dye-labeled substrate for single base extension; and
a fluorescent dye-free substrate for single base extension,
wherein a content ratio of the fluorescent dye-free substrate and the fluorescent dye-labeled substrate is set according to:
(a) ratio of excitation efficiency of the fluorescent dyes to be detected, in a case where at least two types of fluorescent dye are used; and/or
(b) binding-and-uptake efficiency of the substrate to the primers to be used, in a case where at least two types of primer are used.

The kit according to the present invention may typically contain, in addition to the aforementioned constituents, a buffer that constitutes a reaction liquid, enzymes (polymerase, reverse transcriptase, etc.), and a standard sample for calibration. Provision of the primer and the substrate used for the single base extension reaction in the form of kit may enable more quick and convenient gene analysis.

Hereinafter, the present invention will be specifically described with reference to Example. Note that Example is merely intended for explanation of the present invention, and is neither to delineate nor limit the scope of the invention disclosed in the present application.

### EXAMPLES

OncoSpan DNA Reference Standard (Horizon) was used as a standard sample that contains genetic mutations associated with cancer, and EGFR L858, which is a sort of cancer driver gene, was selected as a target gene. Mutation of EGFR L858 is represented by EGFR L858R, indicating single base substitution of the 858-th leucine (L: CUG) with arginine (R: CGG). In this study, also L858Q (Q: glutamine, CAG) and L858P (P: proline, CCG) mutations were examined, in order to verify effectiveness of the present invention on four types of base. First, PCR for cloning was conducted with use of primers L858 Forward (GCAGCATGTCAAGATCACAGATT: SEQ ID NO: 1) and L858 Reverse (CCTCCTTCTGCATGGTATTCTTTCT: SEQ ID NO:2), while employing, as the templates, the aforementioned standard sample that contains EGFR L858 wild-type (EGFR L858WT) and mutant (L858R) genes. The PCR product was transformed into E. coli, cultured in an LB medium, and then amplified by colony direct PCR. A sequence reaction was then allowed to proceed with use of BigDye Terminator Sequencing Kit (Thermo Fisher Scientific Inc.), followed by purification, confirmation of the sequence with a genetic analyzer SeqStudio, and extraction of plasmid. In the cloning of mutant L858Q and L858P, site-directed mutagenesis PCR was conducted on a wild-type plasmid, with use of PrimeSTAR Mutagenesis Basal Kit (Takara Bio Inc.) while employing the primers listed in the table below.

TABLE 1

| Primer | Sequence (5′ → 3′) | SEQ ID NO |
|---|---|---|
| EGFR L858Q | TTTGGGCAGGCCAAACTGCTGGGTGC | 3 |
| Forward | | |
| EGFR L858Q | TTTGGCCTGCCCAAAATCTGTGATCT | 4 |
| Reverse | | |
| EGFR L858P | TTTGGGCCGGCCAAACTGCTGGGTGC | 5 |
| Forward | | |
| EGFR L858P | TTTGGCCGGCCCAAAATCTGTGATCT | 6 |
| Reverse | | |

With use of the extracted plasmid as a template (tumor-derived target gene sequence), 0.2 μM of EGFRL858 primer listed in the table below, 1 U of DNA polymerase, and ddNTPs individually labeled with four types of fluorescent dye (R6G-ddATP, ROX-ddUTP, R110-ddGTP and TAMRA-ddCTP) (PerkinElmer) were mixed, and the mixture was subjected to single base extension reaction in a thermal cycler under conditions of [96° C.×10 seconds->55° C.×5 seconds->60° C.×30 seconds]×25 cycles. The concentration of the target template DNA was conditioned at three levels of 0.1 fmol, 1 fmol, and 10 fmol. The concentration of all the fluorescent dye-labeled substrates (ddNTPs) was set initially to 0.1 μM, the concentration of ROX-ddUTP was then adjusted to 4 μM after specially increasing the quantitativeness according to the present invention (mixing with fluorescent dye-free ddNTPs), to which 1 μM of fluorescent dye R6G-free ddATP, and 10 μM of fluorescent dye R110-free ddGTP were added.

TABLE 2

| | Sequence (5′ → 3′) | Primer | Detectable base | |
|---|---|---|---|---|
| | Non-italic: SP6 promoter (24-mer) | size | Wild | |
| Primer | Italic: target gene sequence | (bp) | type | Mutant |
| EGFR | CAAGCTATTTAGGTGACACTATAGCAGCAT | 52 | T | A, C, G |
| L858-FW2 | GTCAAGATCACAGATTTTGGGC (SEQ ID NO: | | | |
| | 7) | | | |

After the single base extension reaction, dephosphorylation reaction (SAP) was conducted to avoid any interference by an unreacted substrate which is fluorescently-labeled ddNTP. One microliter of SAP was added to 10 μL of the reaction product, and the mixture was allowed to react at 7° C. for one hour, and further at 75° C. for 15 minutes. The thus SAP-treated sample was mixed with a size marker and Hi-Di formamide, the mixture was then treated under heating at 95° C. for 5 minutes, and then subjected to fragment analysis with use of a CE sequencer DS3000 (Hitachi High-Tech Corporation).

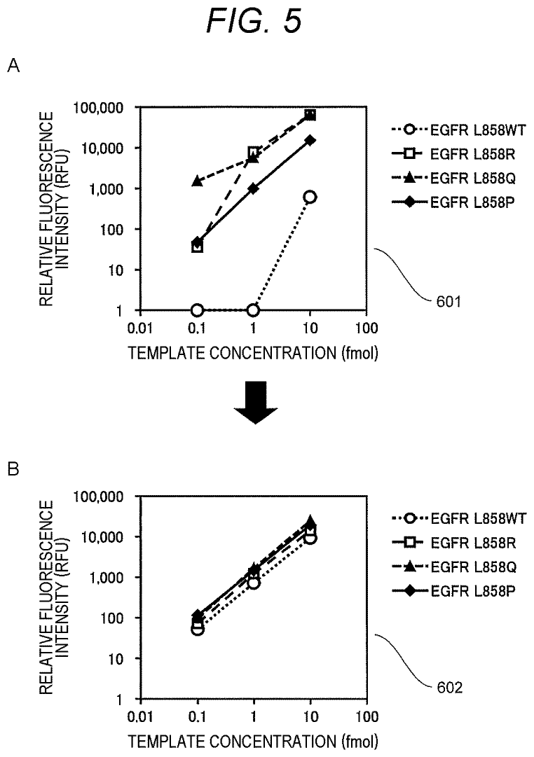

FIG. 5 shows results of template concentration and peak values of fluorescence intensity, before and after the use of the fluorescent dye-free ddNTPs. Abundance of the tumor-derived target gene sequences in terms of relative fluorescence intensity is plotted versus known template concentrations. Use of the fluorescent dye-free ddNTPs resulted in linearity between the abundance and fluorescence intensity, whereby the abundance of the target gene sequence may be determined quantitatively. In an exemplary case with 10 fmol of wild-type EGFR L858WT and 0.1 fmol of mutant EGFR L858Q contained therein, use of the fluorescent dye-free ddNTP successfully showed that the wild-type and the mutant were present in a 100:1 ratio (sensitivity: 1%), by virtue of the quantitativeness of relative fluorescence intensity.

Aforementioned Example has used four types of fluorescent dye, which were rhodamine 6G (R6G), X-rhodamine (ROX), rhodamine 110 (R110), and tetramethylrhodamine (TAMRA). The fluorescent dye in the context of the present invention is, however, not limited thereto, for which any fluorescent dye usually used for labeling nucleic acid probe may be used. The fluorescent dye other than rhodamine derivatives may include fluorescein or fluorescein isothiocyanate (FITC) as a derivative thereof, Alexa 488, Alexa 532, cy3, cy5, and Texas red. The fluorescent dye may be selected, according to the excitation wavelength of laser beam mounted on a capillary electrophoresis apparatus to be used.

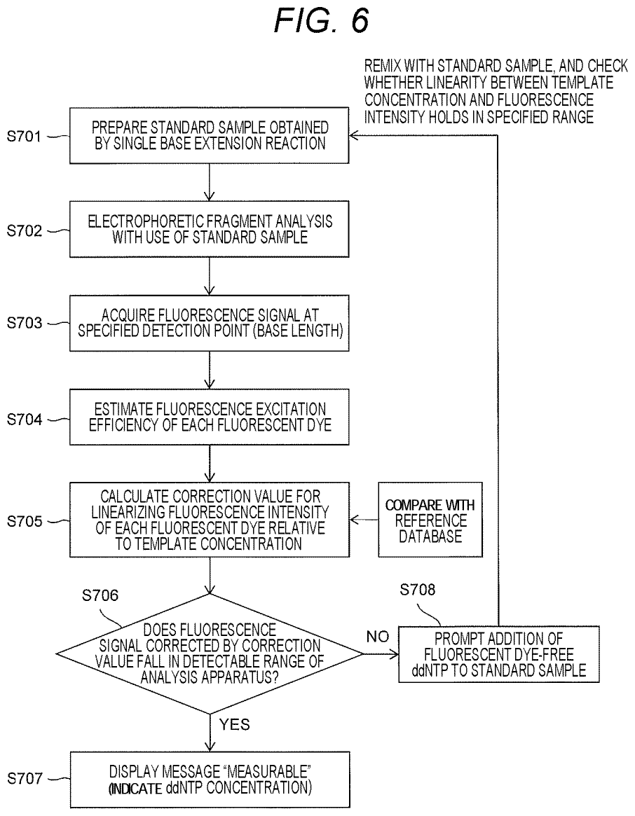

FIG. 6 is a flowchart illustrating an exemplary procedure of processes with use of the gene analysis apparatus and the gene analysis kit for implementing the present invention. The present invention enables quantitative determination of the content ratio of the target base sequences (for example, wild-type and mutant), from the magnitude of the fluorescence intensity. The analysis apparatus herein has a limited range of measurement for fluorescence intensity. Accordingly, pre-treatment may be implemented on the apparatus basis, or with use of the analysis kit, so as to bring the fluorescence intensity into the detectable range of the apparatus.

First, in step S701, a standard sample obtained by the single base extension reaction may be prepared. Next, in step S702, electrophoretic fragment analysis may be implemented with use of a standard sample. The measurement apparatus herein may only be an apparatus adopted to electrophoretic fragment analysis, to which not only capillary electrophoresis apparatus, but also microchannel such as micro-electro-mechanical system (MEMS) may be used. Next, in step S703, a fluorescence signal at a specified detection point (base length) may be acquired, and in step S704, the fluorescence excitation efficiency of each fluorescent dye may be estimated. If the analysis apparatus has installed therein a data holding unit as a mechanism of its main body, such estimation of the fluorescence excitation efficiency may be automated. Next, in step S705, a correction value for linearizing fluorescence intensity (signal) of each fluorescent dye versus the template concentration may be calculated. In this process, while comparing with a reference database, whether or not characteristics of the fluorescent signal has been acquired, or assumed in advance (for example, maximum value, half-value width, peak detection point, etc. of the fluorescence signal) may be checked, and then in step S706, whether or not the fluorescence signal corrected by a correction value falls in the detectable range of the analysis apparatus may be checked. After confirming that the fluorescence intensity falls in the detectable range, and that the content ratio of the target base sequences (wild-type and mutant, for example) can be detected within the range typically from 0.010% to 1%, then in step S707, a message “Measurable” may be displayed. An actual sample may now become measurable. The display in this process may also contain the concentration of the fluorescent dye-free ddNTPs to be added to the actual sample. In contrast, if the fluorescence intensity was determined, in step S706, to fall outside the detectable range, then in step S708, addition of the fluorescent dye-free ddNTP to the standard sample may be prompted. After re-mixing with the standard sample, whether or not the linearity between the template concentration and the fluorescence intensity is obtainable within the specified range may be checked again from step S701. If a data set adapted to the analysis apparatus is prepared in advance, and is allowed for use as an analysis kit, the aforementioned flow of processes may be incorporated on the system basis.

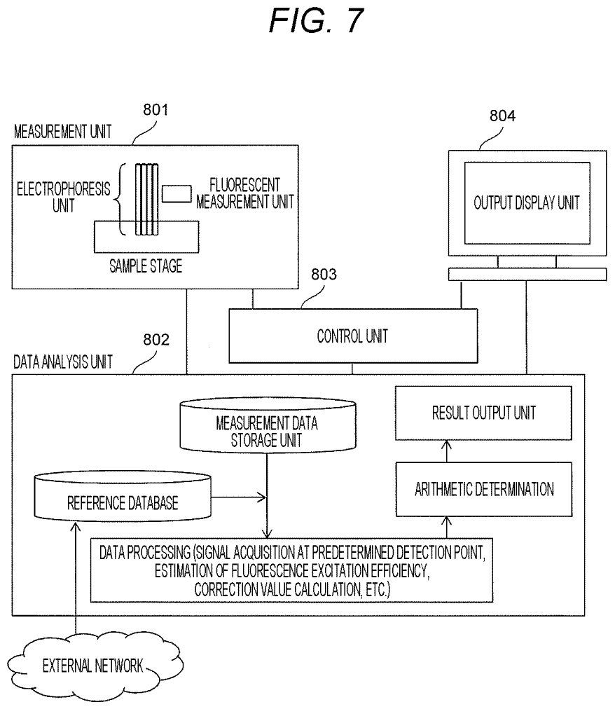

FIG. 7 is a block structural diagram illustrating an exemplary function installed on the gene analysis apparatus of the present invention. The gene analysis apparatus may be mainly constituted by a measurement unit 801, a data analysis unit 802, a control unit 803, and an output display unit 804. In the measurement unit 801, a sample after single base extension may be placed on a sample stage, and fluorescence signals from the sample that flows through an electrophoresis unit in capillary electrophoresis may temporarily be measured with a fluorescence measurement unit. The data analysis unit 802 may have a measurement data storage unit for storing measurement data obtained by the measurement unit 801. A program for processing the data may be implemented by software. As illustrated in the flowchart of FIG. 6, contents of the data processing may typically include acquisition of fluorescence signal at specified detection point (base length), estimation of fluorescence excitation efficiency of each fluorescent dye, and calculation of a correction value for linearizing fluorescence intensity of each fluorescent dye versus template concentration. In this process, reference data of gene sequence preliminarily stored in the data analysis unit 802 may also be used. It may also be acceptable to update the reference data, by transmitting and receiving information to and from an external network. Functional control of the measurement unit 801, the data analysis unit 802 and so forth may be implemented on the software basis, in which a program stored in a memory of the control unit 803 may be interpreted and executed by a processor. Alternatively, the individual structures, functional unit, processing unit, processing means and the like may be implemented on the hardware basis, typically by designing a part of, or all of them for example with an integrated circuit. Programs having the individual functions, and information such as file and database may be stored in a recording device such as memory, hard disk, or solid state drive (SSD), or a recording medium such as IC card, SD card, or DVD. After the data processing, for example, whether or not the fluorescence signal corrected by the correction value falls in the detectable range of the analysis apparatus may be determined by an arithmetic process, and the result may be output on the output display unit 804. The block structural diagram herein illustrates a system integrally given in the form of gene analysis apparatus. Note that the gene analysis method of the present invention is applicable to any form of system if only having functions of the measurement unit 801, the data analysis unit 802, the control unit 803, and the output display unit 804.

The present invention may include various modifications, without being limited to the aforementioned embodiments. For example, the aforementioned embodiments have been detailed merely for easy understanding of the present disclosure, and are not necessarily limited to those having all structures described above. In addition, a part of the structure of a certain embodiment may be replaced with a structure of another embodiment; and a structure of another embodiment may be added to the structure of a certain embodiment. In addition, a part of structure of each embodiment allows addition, deletion or replacement with another structure.

All publications, patents, and patent applications cited herein are incorporated by reference in their entirety.

### REFERENCE SIGNS LIST

101 tumor-derived target gene sequence #1
102 gene sequence #1-selective primer
103 fluorescent dye #1-labeled ddNTP
104 relative fluorescence intensity ascribed to fluorescent dye #1
201 tumor-derived target gene sequence #2
202 gene sequence #2-selective primer
203 fluorescent dye #2-labeled ddNTP
204 relative fluorescence intensity ascribed to fluorescent dye #2
301 tumor-derived target gene sequence #3
302 gene sequence #3-selective primer
303 fluorescent dye #1-labeled ddNTP
401 uptake efficiency of fluorescently-labeled ddNTP increased on gene sequence #1
402 uptake efficiency of fluorescently-labeled ddNTP decreased on gene sequence #3
403 relative fluorescence intensity ascribed to fluorescent dye #1, under increased uptake efficiency of fluorescently-labeled ddNTP on gene sequence #1
404 relative fluorescence intensity ascribed to fluorescent dye #1, under decreased uptake efficiency of fluorescently-labeled ddNTP on gene sequence #3
501 fluorescent dye #1-free ddNTP
502 relative fluorescence intensity ascribed to fluorescent dye #1 indicating abundance of gene sequence #1, in the presence of fluorescent dye #1-free ddNTP
503 relative fluorescence intensity ascribed to fluorescent dye #2 indicating abundance of gene sequence #2
601 results indicating template concentration versus peak values of fluorescence intensity, before use of fluorescent dye-free ddNTPs.
602 results indicating template concentration versus peak values of fluorescence intensity, after use of fluorescent dye-free ddNTPs.
801 measurement unit
802 data analysis unit
803 control unit
804 output display unit

### SEQUENCE LISTING FREE TEXT

SEQ ID NOs: 1 to 7: Artificial (synthetic oligonucleotide)

1. A gene analysis method comprising:
conducting a single base extension reaction with use of a single base extension primer for detecting a target base sequence, and a fluorescent dye-labeled substrate for single base extension;
subjecting a reaction product of the single base extension reaction to electrophoresis; and
measuring mobility in the electrophoresis and fluorescence intensity of the fluorescent dye, and quantifying a content ratio of a plurality of target base sequences from a magnitude of the fluorescence intensity,
wherein in the single base extension reaction, a fluorescent dye-free substrate for single base extension is mixed, and
wherein a mixing ratio of the fluorescent dye-free substrate and the fluorescent dye-labeled substrate is set according to:
(a) ratio of excitation efficiency of the fluorescent dyes to be detected, in a case where at least two types of fluorescent dye are used; and/or
(b) binding-and-uptake efficiency of the substrate to the primers to be used, in a case where at least two types of primer are used.

2. The method according to claim 1, wherein
the fluorescent dye comprises at least two types of fluorescent dye, and
the mixing ratio of the fluorescent dye-free substrate and the fluorescent dye-labeled substrate is set according to the ratio of excitation efficiency of the fluorescent dyes to be detected.

3. The method according to claim 1, wherein
the primer comprises at least two types of primer, and
the mixing ratio of the fluorescent dye-free substrate and the fluorescent dye-labeled substrate is set according to the binding-and-uptake efficiency of the substrate to the primers to be used.

4. The method according to claim 1, wherein
the primer comprises at least two types of primer, and the fluorescent dye comprises at least two types of fluorescent dye, and
the mixing ratio of the fluorescent dye-free substrate and the fluorescent dye-labeled substrate is set according to the ratio of excitation efficiency of the fluorescent dyes to be detected, and according to the binding-and-uptake efficiency of the substrate to the primers to be used.

5. The method according to claim 1, wherein
the plurality of target base sequences comprise a wild-type sequence and a mutant sequence, and
a content ratio of the mutant sequence to the wild-type sequence is quantified within a range from 0.010% to 10%.

6. The method according to claim 1, wherein the primer has a double-stranded DNA tag having an inter-strand crosslink.

7. The method according to claim 1, wherein the electrophoresis is capillary electrophoresis.

8. A gene analysis apparatus comprising:
a measurement unit configured to measure single base extension reaction, electrophoresis, and fluorescence intensity;
a data analysis unit comprising a measurement data storage unit configured to store measurement data obtained by the measurement unit, and a data processing unit; and
a control unit;
wherein the control unit is configured to analyze the measurement data stored in the measurement data storage unit, and to determine a mixing ratio of a fluorescent dye-labeled substrate and a fluorescent dye-free substrate to be used in the single base extension reaction.

9. The apparatus according to claim 8, wherein
the control unit further comprises a reference database configured to store previous measurement data, and
the control unit is configured to compare the measurement data stored in the measurement data storage unit, with the previous measurement data stored in the reference database, and to determine the mixing ratio of the fluorescent dye-labeled substrate and the fluorescent dye-free substrate to be used in the single base extension reaction.

10. The apparatus according to claim 8, further comprising an output display unit.

11. A gene analysis kit comprising:
a single base extension primer for detecting a target base sequence;
a fluorescent dye-labeled substrate for single base extension; and
a fluorescent dye-free substrate for single base extension,
wherein a content ratio of the fluorescent dye-free substrate and the fluorescent dye-labeled substrate is set according to:
(a) ratio of excitation efficiency of the fluorescent dyes to be detected, in a case where at least two types of fluorescent dye are used; and/or
(b) binding-and-uptake efficiency of the substrate to the primers to be used, in a case where at least two types of primer are used.
